(19) United States
(12) Patent Application Publication
(10) Pub. No.: US 2006/0093639 A1
(43) Pub. Date: May 4, 2006

(54) Method and device for destroying body tissue

(76) Inventor: Warren L. Starkebaum, Plymouth, MN (US)

Correspondence Address: SHUMAKER & SIEFFERT, P. A., 8425 SEASONS PARKWAY, SUITE 105, ST. PAUL, MN, 55125, US

(21) Appl. No.: 10/977,338

(22) Filed: Oct. 29, 2004

(51) Int. Cl. A61F 13/00

(52) U.S. Cl. 424/422

(57) ABSTRACT

In general, the invention is directed to devices and methods for destroying targeted body tissue. A hydrogel implant, configured to be implanted in a target tissue, includes an associated chemical agent, such as a proteolytic chemical agent. When implanted in the targeted tissue, the implant expands and discharges the chemical agent, destroying the target tissue. Because the chemical agent is associated with the hydrogel implant, and because the hydrogel implant is less likely to move from its site of implantation, there is reduced risk that the chemical agent will migrate and adversely affect non-targeted tissues.

### FIELD OF THE INVENTION

The invention relates to medical devices implantable in and near a human or animal body.

### BACKGROUND

There are circumstances in which it is desirable to destroy living tissue in a body. In the case of patient having a tumor, for example, the patient may benefit from the destruction of the tumor. In another example, a male patient suffering from a hypertrophic prostate can benefit from the destruction of some tissue in the prostate.

Tissue destruction can be performed by surgical techniques, use of certain drugs, application of ablation, or radiation therapy. Each of these techniques has drawbacks or risks to the patient. Some forms of drug therapy and radiation therapy, for example, have serious side effects, affecting not only the targeted tissue but also other tissues and systems. Surgical intervention, which can focus upon the targeted tissue, carries inherent risks and cause pain and require recovery time.

One technique for destroying targeted tissue is to inject one or more agents that destroy the targeted tissue into the unwanted target tissue. The injection may be accomplished by injection through the skin or by accessing the target tissue by way of a natural anatomical passageway such as a nostril, mouth, urethra, vagina or anus. The injection may be accomplished by obtaining access to the targeted tissue through a surgical procedure.

Some chemicals destroy the tissues they contact. Unfortunately, a risk associated with injection of such chemicals is that the chemicals often do not remain localized in the target tissue. Instead, the chemicals migrate, killing untargeted tissue and causing undesirable side effects. Injection of a chemical such as methanol into the prostate, for example, can relieve symptoms associated with a hypertrophic prostate, but the methanol can also migrate from the site to cause local nerve damage or harm to nearby urethral and bladder structures.

There have been many approaches addressing problems associated with drug delivery to a patient. Many approaches involve use of a hydrogel to deliver therapeutic agents to a patient. Some of the approaches are not suitable for delivery of chemicals that are configured to destroy a targeted tissue, however. Some hydrogel implants are not configured for implantation in a target tissue, but deliver therapeutic agents systemically or in a non-targeted manner. Some hydrogel implants are configured to degrade within the body, and are not capable of delivering certain chemicals that would destroy the target tissue. Proteolytic enzymes that would also destroy a target tissue, for example, would also degrade the implant and thereby reduce the desired localization of the chemical.

Some hydrogel structures that deliver proteolytic agents are not implantable or do not destroy the targeted tissue. U.S. Pat. No. 6,348,042 to Warren, Jr., for example, describes a catheter having an interior surface impregnated with proteolytic enzymes attached with a matrix that can include a hydrogel. The Warren shunt is not directed at destroying tissue proximate to the shunt, but rather is directed to keeping the lumen free from obstructions. Implantable hydrogel delivery devices described in U.S. Patent Application 2003/0203030 are directed to delivering compositions to treat, rather than to destroy, living tissue.

Table 1 below lists documents that disclose some of the many techniques for addressing drug delivery. Some of the documents provide for delivery with hydrogels.

TABLE 1

| Patent Number | Inventors | Title |
|---|---|---|
| 6,749,868 | Brauckman, et al. | Protein stabilized pharmacologi- |
| | | cally active agents, methods for |
| | | the preparation thereof and methods |
| | | for the use thereof |
| Application | Loomis et al. | Bioresorbable hydrogel compositions |
| 2004/0082682 | | for implantable prostheses |
| 6,660,827 | Loomis et al. | Bioresorbable hydrogel compositions |
| | | for implantable prostheses |
| Application | Ashton et al. | Polymeric gel delivery system for |
| 2003/0203030 | | pharmaceuticals |
| 6,639,014 | Pathak et al.. | Multiblock biodegradable hydrogels |
| | | for drug delivery and tissue |
| | | treatment |
| Application | Cleary et al. | Hydrogel compositions |
| 2003/0170308 | | |
| 6,368,598 | D'Amico et al. | Drug complex for treatment of |
| | | metastatic prostate cancer |
| 6,348,042 | Warren, Jr. | Bioactive shunt |
| 5,798,113 | Dionne et al. | Implantable biocompatible immuno- |
| | | isolatory vehicle for delivery of |
| | | selected therapeutic products |

All documents listed in Table 1 above are hereby incorporated by reference herein in their respective entireties. As those of ordinary skill in the art will appreciate readily upon reading the Summary of the Invention, Detailed Description of the Preferred Embodiments and Claims set forth below, many of the devices and methods disclosed in the patents of Table 1 may be modified advantageously by using the techniques of the present invention.

### SUMMARY OF THE INVENTION

The present invention is directed to devices and methods for destroying targeted body tissue. The invention has certain objects. That is, various embodiments of the present invention provide solutions to one or more problems existing in the prior art with respect to addressing destruction of unwanted targeted tissue. The problems include, for example, the risks associated with surgery or radiation, and the side effects associated with existing methods of targeting tissue for destruction. The problems also include migration of a chemical agent that is introduced into the target tissue and that migrates to adversely affect other tissues. Migration can result in damage to or destruction of healthy, non-targeted tissues.

Various embodiments of the present invention have the object of solving at least one of the foregoing problems. In particular, various embodiments of the present invention address the problems associated with destroying target tissues with chemicals while also addressing the problems associated with migration of the chemical agents. In addition, various embodiment of the invention can reduce the pain and recovery time otherwise associated with surgical techniques. In addition, the invention generally avoids the need for radiation exposure of target tissue, and undesirable ancillary exposure of other tissue.

Various embodiments of the invention may possess one or more features capable of fulfilling the objects outlined above. In general, the invention provides for implantation of a hydrogel implant, which is configured to be implanted in a target tissue. A chemical agent, such as a proteolytic chemical agent, is associated with the implant, and the chemical agent is configured to destroy the target tissue.

The hydrogel implant can be loaded with the chemical while in a dehydrated state, for example, or can be loaded with the chemical while in a hydrated state and then desiccated. In the dehydrated state, the hydrogel implant is small and more easily delivered to the target tissue by an implantation device such as a syringe. An exemplary shape for an implant in the dehydrated state is an elongated cylinder-like shape, similar to the shape of a grain of rice. Upon implantation, the hydrogel implant hydrates due to exposure to body fluid, and expands to an enlarged state. In the expanded state, the hydrogel discharges the chemical proximate to its surface. The discharge is generally slow, thereby adversely affecting the target tissues which are in close proximity to the implant, while having less effect upon more remote, non-targeted tissues.

The biocompatible hydrogel implant may deliver any of several chemical agents. One embodiment of the invention supports delivery of proteolytic agents that destroy the target tissue by breaking chemical bonds. In some embodiments of the invention, the hydrogel may be biocompatible but non-biodegradable, thereby resisting degradation by the chemical associated with the implant.

An exemplary procedure that can employ the invention is treatment of hypertrophic prostate, often referred to as benign prostate hypertrophy (BPH). The invention includes a method that comprises implanting one or more hydrogel implants in a prostate of a patient. A chemical agent associated with the implant is configured to destroy tissue of the prostate proximate to the implant, thereby relieving the hypertrophy. The various embodiments of the invention provide for less invasive surgical intervention than other surgical techniques. As a result, the implantations may be performed in less time and with less expense, and with reduced recovery time for the patient. In addition, bulking prostheses implanted according to the invention may be readily removed, if necessary. Also, once the implants are in place, no further maintenance is necessary, as the bulking prosthesis require no electrical power supply and have no coupled moving parts. The techniques of the invention further allow implantation of comparatively large bulking prostheses, which tend to stay in one piece and which tends not to migrate from the site of implantation.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a coronal cross section of anatomical structures surrounding a urethra of a male patient, showing an exemplary placement of a hydrogel implant configured to discharge a chemical agent in accordance with an embodiment of the invention.

FIG. 2 is a cross section of an exemplary syringe that may be used to practice the invention.

FIG. 3 is a cross section of a target tissue receiving hydrogel implants configured to discharge a chemical agent.

FIG. 4 a cross section of the target tissue of FIG. 3 following implantation of the hydrogel implants.

FIG. 5 is a perspective view of an exemplary rice-grain shape of a hydrogel implant configured to discharge a chemical agent in accordance with an embodiment of the invention.

FIG. 6 is a perspective view of an exemplary cylindrical shape of a hydrogel implant configured to discharge a chemical agent in accordance with an embodiment of the invention.

FIG. 7 is a perspective view of an exemplary shape of a hydrogel implant having varying diameters configured to discharge a chemical agent in accordance with an embodiment of the invention.

FIG. 8 is a perspective view of an exemplary substantially spherical shape of a hydrogel implant configured to discharge a chemical agent in accordance with an embodiment of the invention.

FIG. 9 is a perspective view of another exemplary substantially spherical shape of a hydrogel implant configured to discharge a chemical agent in accordance with an embodiment of the invention.

FIG. 10 is a perspective view of an exemplary coiled sheet shape of a hydrogel implant in a dehydrated state.

FIG. 11 is a perspective view of the implant depicted in FIG. 10 in a hydrated form, configured to discharge a chemical agent in accordance with an embodiment of the invention.

### DETAILED DESCRIPTION OF THE PREFERRED EMBODIMENTS

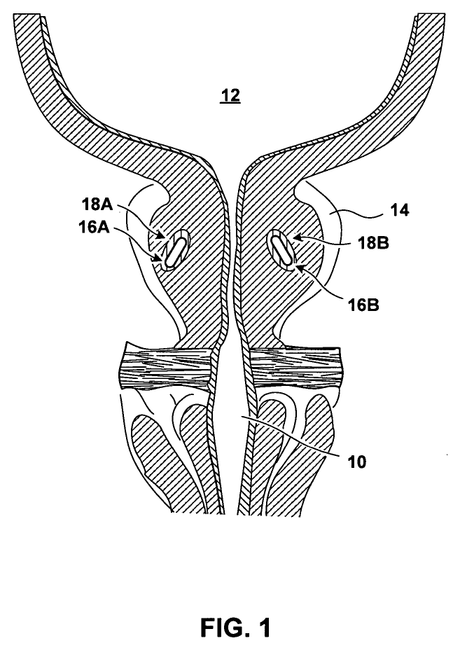

FIG. 1, which depicts an exemplary application of the invention, is a coronal cross section of anatomical structures surrounding a urethra 10 of a male patient. Urethra 10 is a tube, including a wall and a lumen, that extends from the urinary bladder 12 to an external urethral orifice (not shown in FIG. 1). Urethra 10 extends through prostate 14. As depicted in FIG. 1, prostate 14 is enlarged, a condition known as benign prostate hypertrophy (BPH). As a result of BPH, urethra 10 is narrowed as it passes through prostate 14, making it difficult for the patient to urinate.

One or more hydrogel implants 16A, 16B (hereafter 16) have been implanted in prostate 14, in accordance with an embodiment of the invention. Implants 16 are sized for placement within prostate 14. Although FIG. 1 shows two implants 16A and 16B, any number of implants may be used. In one possible deployment, implants 16 are deployed circumferentially around urethra 10, within the medial and lateral prostate lobes. Furthermore, as shown in FIG. 1, implants 16 are implanted in the tissue of prostate 14 at a site that is remote from urethra 10 to avoid damage to the urethra. Implants 16 may be deployed in prostate 14 by any medical technique. A physician may access prostate 14 transrectally, transperionally, transurethrally, or by any other route.

Implants 16 include a chemical agent that destroys target tissue in prostate 14. The chemical agent is associated with implants 16 by, for example, being absorbed in the material that make up implants 16, or by being held in cavities inside implants 16. As a result of exposure to the chemical agent discharged by implants 16 in the example of FIG. 1, target tissue 18A and 18B (hereafter 18) proximate to the external surface of implants 16 has been destroyed and is no longer living tissue. The chemical agent acts on target tissue 18 by any chemical process that destroys target tissue 18. As discussed below, the chemical agent can be any of several proteolytic agents that break chemical bonds and thereby disrupt the activity of target tissue 18.

Deployment of implants 16 remote from urethra 10 promotes destruction of target tissue, i.e., the tissue of prostate 14, and reduces the likelihood that the chemical agent will act upon useful tissue, such as urethra 10. Mere deployment of the chemical agent into prostrate 14 without an implant results in a risk of migration of the chemical agent to useful tissue, such as urethra 10 or bladder 12. Delivery of the chemical agent via hydrogel implants 16, however, reduces the risk of chemical migration. Instead of being free to move within the tissue, the chemical agents emit from implants 16, and are therefore constrained by the shapes and sizes of implants 16. Implants 16 are less likely to migrate than an unconstrained chemical agent.

Once target tissue 18 is destroyed, target tissue 18 is further broken down and removed or reabsorbed by the patient's system. In the prostate, it has been observed that destruction of tissue in prostate 14 leads to reduced swelling and relief of the symptoms of BHP. As target tissue 18 is removed from proximity to implants 16, additional tissue of prostate 14 may come in proximity to implants 16 and be affected by the chemical agent, thereby reducing swelling further. Although implantation of implants 16 initially increases the volume of prostate 14, it is believed that the destruction of target tissue 18 eventually leads to an overall decrease in volume.

In a typical implementation of an implant, the implant imparts the chemical agent to the proximate tissue for a limited time. After a period, the implant no longer delivers the chemical agent. In particular, the implant is supplied with an amount of the chemical agent that is exhausted over time by emission into surrounding tissue. The implants are generally biocompatible and in many cases it is not necessary to remove the implants. In the case of one or more implants deployed in the prostate to treat BPH, for example, it may be desirable to remove the implants after a period of time.

Implants 18 are formed from any suitable hydrogel material. “Suitability” of a hydrogel is a function of many factors, including biocompatibility, desired flexibility, desired expansion, and the like. Suitability is also a function of the interaction between the hydrogel and the chemical agent. Some hydrogels break down in the presence of enzymes that may be naturally present in the body of the patient, and it may not be desirable to use such a hydrogel to deliver a chemical agent that will cause the hydrogel to break down. Some embodiments of the invention therefore employ a non-biodegradable hydrogel that resists degradation in the presence of the chemical agent that is associated with the implant.

Hydrogel materials that are believed to have wide applicability are the polyacrylonitrile copolymers as described in U.S. Pat. Nos. 4,943,618 and 5,252,692, which are incorporated herein by reference. By controlling relative amounts of the copolymers, it is often possible to regulate physical qualities of the hydrogel such as flexibility and amount of expansion. It is also possible to regulate the rate at which the hydrogel implant discharges the chemical agent. In the case of chemical agents that diffuse through a hydrogel barrier to the external surface of the implant, for example, the diffusion rates of chemicals through hydrogel compositions can be determined by experimentation, and a combination of chemical and hydrogel compositions can be selected to produce a desired rate of chemical discharge.

In general, hydrogels can assume a dehydrated state and a hydrated state. A hydrogel implant in its dehydrated state is generally substantially smaller than the implant in its hydrated state. A hydrogel implant in its dehydrated state, when placed into the body of a patient, absorbs water from the body and swells, assuming a hydrated state. During implantation, the hydrogel implant is often in the dehydrated state, where it is more miniaturized and more easily implanted by a syringe or endoscope.

The chemical agent that is associated with the hydrogel can be any of several chemical agents that destroy living tissue. The chemical agent can include a proteolytic agent, but the invention is not limited to proteolytic agents. A partial listing of commercially available proteolytic agents is shown below.
Achromopeptidase
Aminopeptidase
Ancrod
Angiotensin Converting Enzyme
Bromelain
Calpain
Calpain I
Calpain II
Carboxypeptidase A
Carboxypeptidase B
Carboxypeptidase G
Carboxypeptidase P
Carboxypeptidase W
Carboxypeptidase Y
Caspase
Caspase 1
Caspase 2
Caspase 3
Caspase 4
Caspase 5
Caspase 6
Caspase 7
Caspase 8
Caspase 9
Caspase 10
Caspase 13
Cathepsin B
Cathepsin C
Cathepsin D
Cathepsin G
Cathepsin H
Cathepsin L
Chymopapain
Chymase
Chymotrypsin, α-
Clostripain
Collagenase
Complement C1r
Complement C1s
Complement Factor D
Complement factor I
Cucumisin
Dipeptidyl peptidase IV
Elastase, leukocyte
Elastase, pancreatic
Endoproteinase Arg-C
Endoproteinase Asp-N
Endoproteinase Glu-C
Endoproteinase Lys-C
Enterokinase
Factor Xa
Ficin
Furin
Granzyme A
Granzyme B
HIV Protease
Igase
Kallikrein tissue
Leucine Aminopeptidase (General)
Leucine aminopeptidase, cytosol
Leucine aminopeptidase, microsomal
Matrix metalloprotease
Methionine Aminopeptidase
Neutrase
Papain
Pepsin
Plasmin
Prolidase
Pronase E
Prostate Specific Antigen
Protease, Alkalophilic from Streptomyces griseus
Protease from Aspergillus
Protease from Aspergillus saitoi
Protease from Aspergillus sojae
Protease (B. licheniformis) (Alkaline)
Protease (B. licheniformis) (Alcalase)
Protease from Bacillus polymyxa
Protease from Bacillus sp
Protease from Bacillus sp (Esperase)
Protease from Rhizopus sp.
Protease S
Proteasomes
Proteinase from Aspergillus oryzae
Proteinase 3
Proteinase A
Proteinase K
Protein C
Pyroglutamate aminopeptidase
Renin
Rennin
Streptokinase
Subtilisin
Thermolysin
Thrombin
Tissue Plasminogen Activator
Trypsin
Tryptase
Urokinase

The invention is not limited to the particular proteolytic agents listed above.

There are a number of techniques whereby the chemical agents can be encased within, chemically combined with, incorporated into, or otherwise associated with the hydrogel implants. In some embodiments, the chemical agent can be incorporated into the hydrogel while both the agent and the hydrogel are in a dehydrated form. Some chemical agents can be turned into a powder by techniques such as lyophilization, and in the dehydrated state, are inactive. When the hydrogel enters a hydrated state, such agents likewise become hydrated, and be activated thereby.

Another technique for associating a chemical agent with a hydrogel implant is to mix the hydrogel and the agent in a hydrated state, and desiccate the substances together. The hydrated hydrogel can also be immersed in a concentrated solution of the proteolytic agent, which allows the proteolytic agent to be absorbed by the hydrogel, followed by dehydration. A further technique is to apply a coating of the chemical agent to the hydrogel while the hydrogel is in a dehydrated state. An additional technique involves insertion of the chemical agent into a cavity such as by injection of the chemical agent into the implant while the implant is in a hydrated state. The agent can exit via paths such as diffusion. Some chemical agents transport through interstices in the hydrogel, and diffuse from the hydrogel by following a concentration gradient.

The quantity of chemical agent associated with a particular hydrogel implant is restricted by factors such as the size of the implant or the physical characteristics of the hydrogel. As a result, the effect of the chemical associated with the hydrogel implant is generally limited in time. Over time, the chemical is depleted, degraded or otherwise loses its effectiveness, and the implant becomes inert.

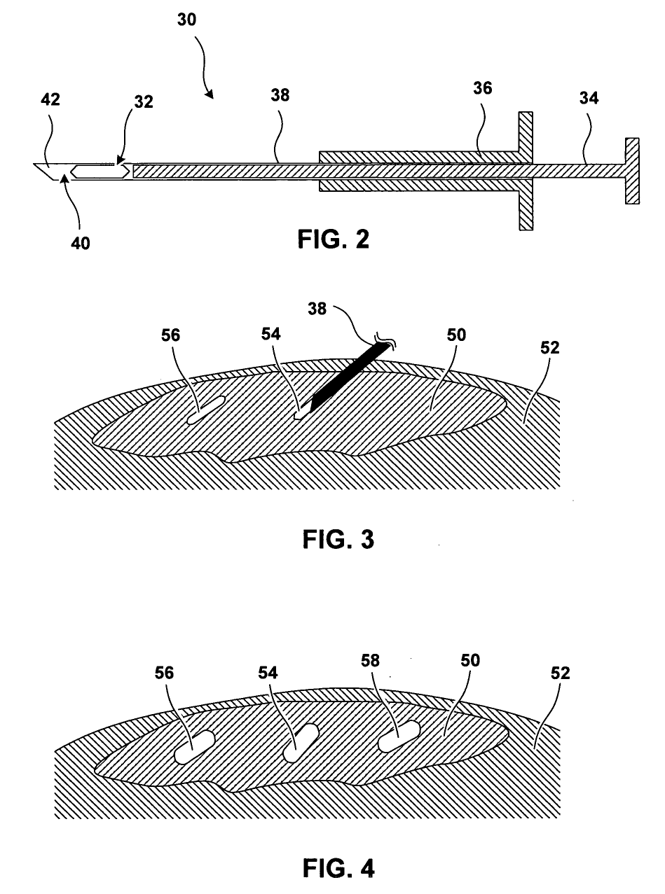

FIG. 2 is a cross-sectional diagram of an exemplary implantation device 30 that can implant a hydrogel implant 32 having a chemical agent. Device 30 comprises a syringe, which includes a plunger member 34, a body member 36 and a hollow needle 38 having a lumen 40. Needle 38 is fixedly coupled to body member 36, while plunger member 34 is free to move in lumen 40. Lumen 40 of needle 38 has been enlarged to show implant 32, in a miniature dehydrated state, disposed in lumen 40.

As depicted in FIG. 2, hydrogel implant in the dehydrated state is an elongated implant, shaped substantially like a grain of rice. Dimensions of implant 32 in the dehydrated state can be approximately one to six millimeters in diameter and approximately five to ten millimeters in length, but the invention encompasses other shapes and dimensions as well. For example, the invention encompasses embodiments in which a hydrogel implant includes one or more hydrogel anchors or fixation structures that engage the target tissue upon implantation and restrict migration of the implant. Examples of fixation structures include protrusions, troughs and the like. When the implant is in the dehydrated state, the fixation structures are not prominent and do not interfere with delivery of the implant with a needle. When the implant enters a hydrated state, the implant swells and the fixation structures become more prominent.

The amount of swelling that accompanies hydration can ordinarily be regulated by adjusting the relative quantities of copolymers in the chemical composition of the implant, or by physically processing the hydrogel. Stretching an implant during formation, for example, can affect the degree of expansion in particular directions. A typical diameter expansion for implant 32 from the dehydrated form to the hydrated form can be four to five times, with less expansion of length.

Distal end 42 of needle 38 includes a sharp point that can pierce tissue such as the skin, the mucosa of the gastrointestinal tract, a body organ or a tissue mass. Distal end 42 further includes an opening through which implant 32 may be expelled from lumen 40 by depressing plunger member 34 with respect to body member 36.

The invention is not limited to implantations with device 30. Other devices, such as an endoscope, trocar, or catheter, can be used to deploy one or more implants in target tissue.

FIGS. 3 and 4 are cross-sections of a mass of localized target tissue 50, surrounded by healthy tissue 52. Target tissue 50 can be any tissue, such as tumor or hypertrophic prostate tissue. For purposes of illustration, target tissue 50 is presented as a mass of cells forming a neoplasm disposed below the skin of a patient. It is desirable that target tissue 50 be destroyed.

As shown in FIG. 3, needle 38 of device 30 penetrates into target tissue 50 and deposits a hydrogel implant 54 having a chemical agent. Additional hydrogel implants 56, 58 can be deposited in target tissue 50 in a similar fashion.

As shown in FIG. 4, implants 54, 56, 58 are deployed remote from healthy tissue 52. Implants 54, 56, 58 generally migrate little, and therefore tend to remain in target tissue 50. Chemical agents from implants 54, 56, 58 destroy target tissue 50, and the natural body processes dispose of the destroyed tissue. Although not shown in FIG. 4, hydrated implants include one or more protrusive fixation structures that engage the target tissue and restrict migration of the implants. Implants 54, 56, 58 may be removed at the discretion of the patient and the patient's physician, or may remain implanted.

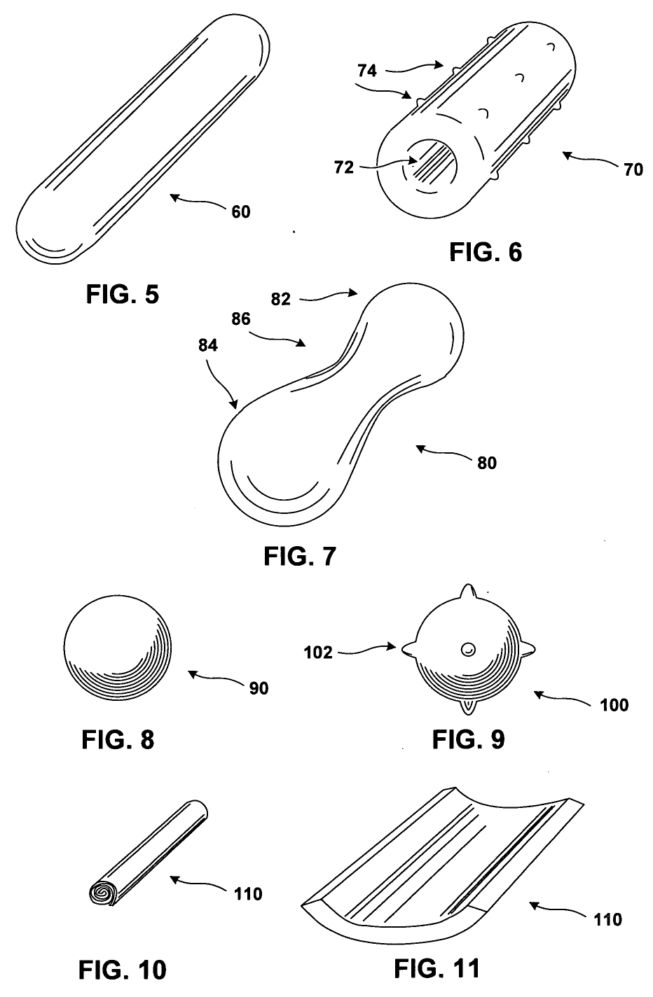

FIGS. 5-10 depict various shapes of implants that can be configured to discharge a chemical agent. FIG. 5 depicts a rice-grain shaped implant 60 having a substantially circular cross-section. Implant 60 can be about one-half to two millimeters in diameter and two to ten millimeters in length when in the dehydrated form. In the hydrated form, the diameter can expand many times, such as four to five times. The length can expand to a greater or lesser degree. The expansion can be regulated by the chemical composition of implant 60. The expansion can be regulated by the processing history, such as the stretching of the implant during formation.

FIG. 6 shows a cylindrical implant 70 having a ring-shaped cross-section. In its expanded hydrated form, implant 70 includes a bore 72. As shown in FIG. 6, bore 72 is hollow. In some embodiments of the invention, bore 72 is closed at each end and includes a chemical agent. The dimensions of implant 70 may be comparable to those of implant 60, i.e., about one-half to two millimeters in outer diameter and 2 to 10 millimeters in length when in the dehydrated form.

In the embodiment shown in FIG. 6, implant 70 includes a plurality of protrusive fixation structures 74. The structures are in the form of bumps that engage the target tissue and restrict migration of implant 70.

FIG. 7 depicts a peanut-shaped implant 80 in the hydrated form. In the dehydrated form, the dimensions of implant 80 may be comparable to those of implants 60 and 70. As shown in FIG. 7, implant 80 has a substantially circular cross-section with varying diameters. Thick portions 82, 84 have a larger diameter than thin portion 86. Thin portion 86 may have half the diameter of thick portions 82, 84, for example.

FIGS. 8 and 9 show implants 90, 100 that are substantially spherical. In the dehydrated state, implants 90, 100 can have dimensions of one-half to two millimeters in diameter. In the hydrated state, implants 90, 100 can be one to six millimeters in diameter. Implant 100 includes protrusive fixation structures 102 that engage the target tissue and restrict migration of implant 100.

FIGS. 10 and 11 show is a perspective view of an implant 110 that has a sheet shape. FIG. 10 shows implant 110 in a dehydrated state and coiled. In the dehydrated and coiled state, implant 110 can be implanted in target tissue via an implantation device such as device 30 shown in FIG. 2.

FIG. 11 is a perspective view of implant 110 in a hydrated form. As implant 110 hydrates, implant 110 uncoils. Implant 110 can be about one-half to two millimeters thick in hydrated form, and two to ten millimeters in length and width.

By deployment of a chemical agent with a hydrogel implant, such as described above, the chemical agent is deployed in a localized fashion, proximate to the exterior surface of the implant. Conventional approaches, in which a chemical agent is injected into target tissue, can result in migration of the chemical agent and harm to non-targeted tissues.

The invention encompasses implants that deliver one or more chemical agents in a targeted manner. Although some conventional approaches involve use of a hydrogel to deliver therapeutic agents to a patient, those approaches are not suitable for delivery of chemicals to destroy a targeted tissue. In contrast to many hydrogel implants, the implants described herein are generally configured to be implanted in the target tissue. As a result, the implants deliver a chemical to the target tissue directly, rather than in a systemic or in a non-targeted manner. In addition, the rate of discharge of the chemical from the implant can be controlled, further reducing the risk of harmful concentrations migrating to non-targeted tissues.

The preceding specific embodiments are illustrative of the practice of the invention. It is to be understood, therefore, that other expedients known to those skilled in the art or disclosed herein may be employed without departing from the invention or the scope of the appended claims. For example, the invention is not limited to an implant having the shape and illustrative dimensions described above. Although the invention is described as useful in application with the prostate, the invention is not limited to that application. Furthermore, the invention can be deployed via implantation techniques in addition to those described above. The invention further includes within its scope methods of making and using the implants described above.

In the appended claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

1. A device comprising:
an implant comprising a hydrogel configured to be implanted in a target tissue; and
a chemical agent associated with the implant, the chemical agent configured to destroy the target tissue.

2. The device of claim 1, wherein the chemical agent is a proteolytic chemical agent.

3. The device of claim 1, wherein the hydrogel is non-biodegradable.

4. The device of claim 1, wherein the hydrogel implant is substantially rice grain-shaped when the implant is in a dehydrated state.

5. The device of claim 4, wherein the implant has a diameter of approximately one to six millimeters, and a length of approximately five to thirty millimeters when in a substantially dehydrated state.

6. The device of claim 4, wherein the implant has a diameter of approximately two to 30 millimeters when in a substantially hydrated state.

7. The device of claim 1, wherein the implant comprises a cavity configured to receive the chemical agent.

8. The device of claim 1, wherein the hydrogel comprises a polyacrylonitrile copolymer.

9. The device of claim 1, wherein the implant is sized for placement within a prostate of a patient.

10. The device of claim 1, wherein the hydrogel implant is substantially spherical when the implant is in a hydrated state.

11. The device of claim 10, wherein the implant has a diameter of approximately one to six millimeters when in the hydrated state.

12. The device of claim 1, wherein the hydrogel implant is substantially peanut-shaped when the implant is in a hydrated state.

13. The device of claim 1, wherein the hydrogel implant is substantially sheet-shaped when the implant is in a hydrated state.

14. The device of claim 13, wherein the implant is coiled in a dehydrated state and coiled in the hydrated state.

15. The device of claim 13, wherein the implant has a thickness of approximately one-half to two millimeters and a length of approximately two to ten millimeters when in the hydrated state.

16. A method comprising:
implanting a hydrogel implant in target tissue in a patient,
wherein a chemical agent is associated with the implant,
wherein the chemical agent is configured to destroy at least a portion of the target tissue, and
wherein the hydrogel implant is configured to deliver the chemical agent to the target tissue following the implantation.

17. The method of claim 16, wherein the hydrogel implant is a first hydrogel implant, the method further comprising:
implanting a second hydrogel implant in the target tissue,
wherein the chemical agent is associated with the second implant.

18. The method of claim 16, wherein the target tissue comprises hypertrophic prostate tissue.

19. The method of claim 18, wherein implanting the hydrogel implant in the target tissue comprises:
accessing the prostate by at least one of a transrectal route, a transperional route or a transurethral route; and
implanting the implant in the prostate tissue.

20. The method of claim 16, wherein the target tissue comprises a tumor.

21. A device comprising:
an implant means configured to discharge a chemical agent means to target tissue proximate to the implant means; and
the chemical agent means configured to destroy the target tissue,
wherein the implant means comprises a hydrogel configured to be implanted in the target tissue.

22. The device of claim 21, wherein the hydrogel is non-biodegradable.

23. The device of claim 21, wherein the chemical agent means comprises a proteolytic chemical agent.

24. The device of claim 21, wherein the implant means in a hydrated state is substantially of one of a rice grain-shape, a spherical shape, a peanut shape, or a sheet shape.

25. The device of claim 21, wherein the implant means comprises a cavity configured to receive the chemical agent means.

26. The device of claim 21, wherein the hydrogel comprises a polyacrylonitrile copolymer.
